(19) United States
(12) Patent Application Publication
(10) Pub. No.: US 2022/0378644 A1
(43) Pub. Date: Dec. 1, 2022

(54) APPARATUS FOR STORAGE AND TRANSPORTATION OF AN EXOSKELETON

(71) Applicant: Dignity Health, San Francisco, CA (US)

(72) Inventor: Trent Maruyama, San Francisco, CA (US)

(21) Appl. No.: 17/771,329

(22) PCT Filed: Oct. 30, 2020
(86) PCT No.: PCT/US2020/058404

§ 371 Date: Apr. 22, 2022

Related U.S. Application Data
(60) Provisional application No. 62/927,982, filed on Oct. 30, 2019.
(60) Provisional application No. 62/946,642, filed on Dec. 11, 2019.

(51) Int. Cl. A61H 1/02; A61G 5/10; A61G 5/12; B25J 9/00; A61B 5/00; A61G 5/08

(52) U.S. Cl. CPC A61H 1/0255; A61G 5/101; A61G 5/128; B25J 9/0006; A61B 5/6831; A61G 5/085

(57) ABSTRACT

Disclosed herein is a system/apparatus for storing, use in therapy, and transporting a (bionic) exoskeleton. The apparatus includes a frame comprising a plurality of legs, a seat, and a back which are operable for being moved between one or more configurations. The seat of the frame defines a seat surface configured for engagement with an exoskeleton. Furthermore, the back of the frame is operably engaged with the seat such that the back of the frame may be folded over, into, or extended from the frame to reduce an overall profile size of the apparatus in a collapsed or folded configuration. The apparatus may further include a pair of footrests defined along the legs that rotate in a lateral direction relative to the frame for the patients ease of transition into the exoskeleton. The frame also includes a locking mechanism that aids to from a storage configuration or a ready configuration.

### CROSS REFERENCE TO RELATED APPLICATION

This document is a PCT patent application that claims benefit to U.S. provisional patent application Ser. No. 62/927,982 filed on Oct. 30, 2019, and further claims benefit to U.S. provisional patent application Ser. No. 62/946,642 filed on Dec. 11, 2019; all of which is herein incorporated by reference in its entirety.

### FIELD

The present disclosure generally relates to systems and accessories associated with an exoskeleton; and in particular, to a system/apparatus for storing, use in therapy, and transporting a (bionic) exoskeleton.

### BACKGROUND

The use of exoskeletons (e.g., bionic, powered, etc.) in the application of physical therapy for paraplegic and otherwise physically disabled patients is a burgeoning practice. However, bionic exoskeletons can be heavy and awkward in shape, making them difficult to store, bring out, and put away between physical therapy sessions. In addition, bionic exoskeletons can be very expensive and must be easily portable, as many facilities may only have access to a few exoskeletons to be used across many patients. During physical therapy sessions, the current practice is to store the exoskeleton using any means possible, including the use of office chairs and wheelchairs. However, these simple chairs are easily torn or broken by the heavy metallic frame of the exoskeleton, and the cumbersome limbs of the exoskeleton can make it difficult to store or carry the exoskeleton.

It is with these observations in mind, among others, that various aspects of the present disclosure were conceived and developed.

### BRIEF DESCRIPTION OF THE DRAWINGS

FIG. 1A a simplified block diagram illustrating an exemplary exoskeleton and one embodiment of an apparatus for the storage and transportation of the exemplary exoskeleton.

FIG. 1B is a simplified block diagram of the exemplary exoskeleton engaged to the apparatus of FIG. 1A for storage and/or transportation.

FIG. 2A is a perspective view of another embodiment of an apparatus for the storage and transportation of an exoskeleton.

FIGS. 2B and 2C are aerial views of first and second positions for a pair of footrests of the apparatus of FIG. 2A.

FIG. 3A is an isometric view of another embodiment of an apparatus for the storage and transportation of an exoskeleton.

FIG. 3B is a side view of the embodiment of FIG. 3A in a storage configuration.

FIG. 3C is a side view of the embodiment of FIG. 3A in a partially folded configuration.

FIG. 3D is an isometric view of the embodiment of FIG. 3A in the partially folded configuration.

FIG. 3E is a side view of the embodiment of FIG. 3A in a fully collapsed configuration.

FIG. 3F is a side view of the embodiment of FIG. 3A with a handle and wheels deployed for transport (transport configuration).

Corresponding reference characters indicate corresponding elements among the view of the drawings. The headings used in the figures do not limit the scope of the claims.

### DETAILED DESCRIPTION

Various embodiments of an apparatus for storage and transportation of an exoskeleton, including an associated system and methods thereof are described herein. In some embodiments, the apparatus includes a frame comprising a plurality of legs, a seat, and a back which are operable for being moved between one or more configurations. The seat of the frame defines a seat surface configured for engagement with an exoskeleton. Furthermore, the back of the frame is operably engaged with the seat such that the back of the frame may be folded over, into, or extended from the frame to reduce an overall profile size of the apparatus in a collapsed or folded configuration. Some embodiments of the apparatus may further include a pair of footrests defined along the legs that rotate in a lateral direction relative to the frame for the patient's ease of transition into the exoskeleton. In other embodiments, the footrests may be positioned/foldable along the legs in a manner so as not to interfere with the positioning of the exoskeleton or inhibit the movement of the exoskeleton while in use with the person or in transition to and from the exoskeleton. In some embodiments the frame includes a locking mechanism that locks or transitions one or more of the aforementioned components of the frame to or from a storage configuration or a ready configuration. In addition, the frame is operable for storing itself or the exoskeleton in a compact and portable position in a “transport” configuration or for bearing the weight of a patient and the exoskeleton as the patient is able to sit in the exoskeleton while the exoskeleton engaged to the frame in a “sit-to- and-from-stand” configuration.

### Introduction: Technical Challenges/Problems

Use of exoskeletons has gained considerable popularity in assisting patients with lower-limb paralysis through gait therapy and related methods. To operate, exoskeletons require a surface for patients to go unencumbered from a sitting position to a standing position. Transport and storage of exoskeletons is paramount. At present, clinicians generally use basic chairs for both transport and storage of the exoskeletons. However, there are various known chair-exoskeleton compatibility issues which result in damage to the chair and/or the exoskeleton. In addition, technology is lacking or insufficient that would potentially accommodate transport and storage of an exoskeleton, and also assist the patient generally with movement from the sitting position to the standing position. Accordingly, clinicians have limited options available with respect to technology sufficient to meet the subject needs associated with exoskeletons and use thereof.

### Embodiments Responsive to Technical Challenges/Problems

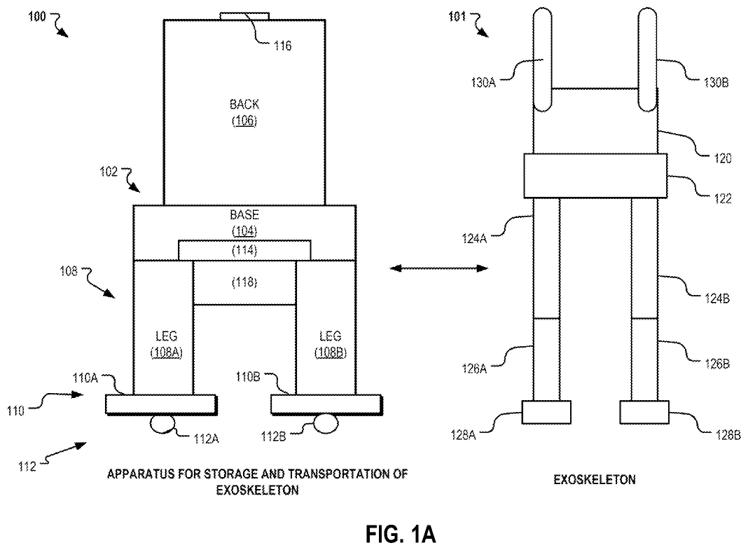

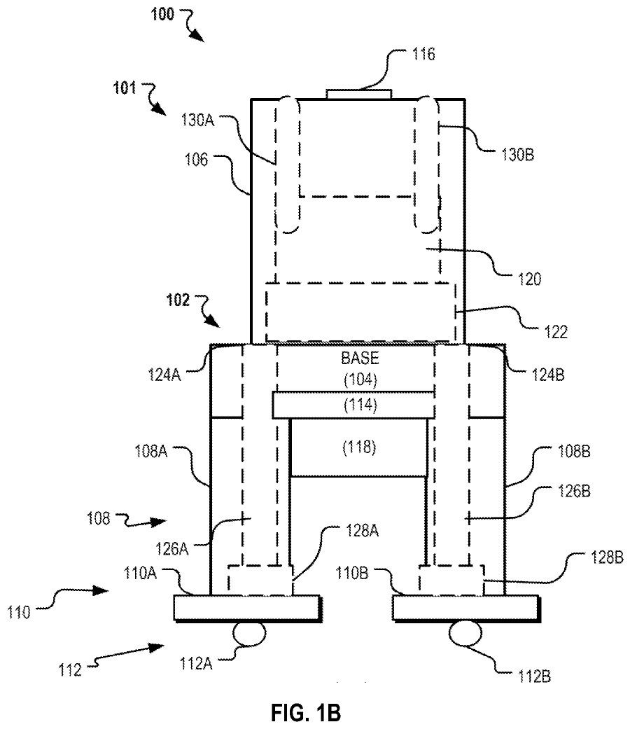

Referring to FIG. 1A, one general embodiment of an apparatus 100 for storage and transportation of an exoskeleton 101 is shown. The apparatus 100 includes a body 102 defining a base 104, and a back 106 is positioned along a top edge of the base 104 as shown. In a deployed or transport configuration, the back 106 extends vertically from the base 104 to support a trunk or frame of an exoskeleton as further described herein. In some embodiments, both the base 104 and the back 106 may define a general rectangular shape, and may be at least partially planar to accommodate engagement with various components of an exoskeleton. Other shapes of the body 102 are contemplated depending on the functionality of the apparatus 100 and how the components of the apparatus 100 display or fold.

As further indicated, a plurality of legs 108, designated leg 108A and leg 108B, may extend from the base 104 along a side of the base 104 opposite the back 106. In general, the plurality of legs 108 may define elongated members separating a ground surface from the base 104. The plurality of legs 108 may include one or more legs, and may take the form of any variety of shapes suitable for positioning the base 104 a predetermined height (which may be adjustable) above the ground/surface, and suitable for supporting a predetermined exoskeleton weight, and possibly a weight of a human in combination with the weight of an exoskeleton. The plurality of legs 108 are shown as discrete members; however it is contemplated that a single contiguous leg component may be employed for some applications.

The apparatus 100 may include various forms of a footrest assembly 110 depending upon the exoskeleton/for which it is intended. In some embodiments, a footrest 110A is defined, in communication with, or otherwise positioned along the leg 108A opposite the base 104; and a footrest 1108 is defined, in communication with, or otherwise positioned along the leg 108A opposite the base 104. Alternatively, however, the footrest assembly 110 may include a contiguous single member horizontally aligned along the plurality of legs 108 (which may fold up vertically). The shape and form of the footrest assembly 110 may be modified to accommodate different exoskeleton forms.

The apparatus 100 further includes a motion mechanism 112 for movement along a ground surface. In some embodiments, the motion mechanism 112 is defined by a plurality of wheels, such as wheel 112A positioned along the bottom of the footrest 110A, and wheel 112B positioned along the bottom of the footrest 1108 (or a total of three or more wheels, where the plurality of legs is greater than two). While wheels are depicted, the motion mechanism 112 may include, by non-limiting examples, caterpillar tracks, pedrail wheels, spheres, a sled or travois, and any other such wheel alternatives. A locking mechanism (not shown) may be integrated with the motion mechanism 112 to impede movement of the apparatus 100 when desired. For example, a brake assembly (not shown) or other such device may be implemented so that when engaged the wheel 112A and the wheel 112B are each prevented from rotating to temporarily maintain the apparatus 100 in a fixed position along a surface (to, e.g., facilitate removal of an exoskeleton therefrom, or enable a person to sit along the base 104 to engage an exoskeleton). A step button or lever (not shown) may further be integrated along the apparatus 100 to engage/disengage the wheel 112A and/or the wheel 112B.

As further shown, the apparatus 100 may include any other components that may facilitate transportation and storage of an exoskeleton, including, but not limited to a storage compartment 114 for storing removable portions of an exoskeleton or tools for assembly thereof, a handle 116 for guiding movement of the apparatus along a surface, and the like. A cushion material, which may be tear-resistant (not shown), may be mounted along the base 104 and the back 106 to provide comfort to an individual utilizing the apparatus 100 to don an exoskeleton.

In some embodiments, the apparatus 100 may include any number of joints or hinges (not shown) to facilitate manipulation of the apparatus to a folded or collapsed configuration. For example, the back 106 may be configured to fold flat along the base 104, and the legs 108 may be configured to fold along the bottom of the base 104 opposite the back 106. In addition, the footrest assembly 110 may be foldable relative to the plurality of legs 108 (e.g., the footrest 110A may fold up along the leg 108A).

In the configuration shown, the apparatus 100 may define a general chair/wheelchair shape configuration. However, the apparatus 100 may include any number or arrangement of configuration change components 118 such as electric motors, pneumatics, levers, hydraulics, or actuators to facilitate movement or rotation of the components of the apparatus 100 relative to one another. For example, in some embodiments, the plurality of legs 108 (and/or the height/length of the apparatus 100 generally) may be extendable and/or retractable using any number of actuators, mechanically, and/or electromechanically, or the configuration change components 118 may be engaged to rotate the base 104 relative to the back 106 to aid with seating of an individual along the apparatus 100. The configuration change components 118 may include electrical components interconnected by any number of conductive members (not shown), or the configuration change components 118 may be devoid of electrical components to reduce the weight and complexity of the apparatus 100 and/or facilitate folding.

Numerous related features are contemplated. For example, the apparatus 100 may comprise aluminum, titanium, composites, carbon fiber, or any number or combination of such materials to provide a lightweight yet durable structure. In some embodiments, aspects of the apparatus 100 may be formed using tubing (e.g., Comodita Brava rollator tubing) with an internal support, such as an I-beam. A seat cover may be formed along the apparatus 100 and may comprise Dyneema fabric or other similar material, and may be replaceable. In general, the apparatus 100 is configured to support at least a 220 pound person and an exoskeleton ranging from 10 to 100 pounds.

As further shown in FIG. 1A and in FIG. 1B, the apparatus 100 is configured to receive the exoskeleton 101 for enhanced storage and transportation; and when engaged to the apparatus 100, the exoskeleton 101 is more easily donned by an individual for use. The exoskeleton shown is merely exemplary, as it is well-known to those skilled in the art that an exoskeleton may take different forms and include different features or components. In general however, the exoskeleton 101 shall be described with components that are common to most forms. Accordingly, the exoskeleton 101 includes a plurality of limb components such as a trunk 120, a midsection 122 in communication with the trunk 120 (often referred to as a hip joint), and an upper leg member 124A and an upper leg member 124B extending from the midsection 122. The exoskeleton 101 further includes a lower leg member 126A in communication with the upper leg member 124A, and a boot 128A defined along the lower leg member 126A as shown. Similarly, a lower leg member 126B is in communication with the upper leg member 124B, and a boot 128B is defined along the lower leg member 126B as shown. The exoskeleton 101 may further include a plurality of straps, designated strap 130A and strap 130B for engaging along a shoulder of an individual.

The exoskeleton 101 is not limiting and may include any number of electric motors, pneumatics, levers, hydraulics, actuators, or electrical/mechanical/electro-mechanical components to accommodate movement of the limbs of the device. The exoskeleton 101 may also include additional structural features such as a thigh brace, interface devices, or other such components. The shape and configuration depicted is merely exemplary.

As shown in FIG. 1B, the exoskeleton 101 may be positioned along the apparatus 100 for storage and transportation, such that at least a portion of the midsection 122 rests along the base 104, the trunk 120 (and straps 130A and 130B) rests along the back 106, and at least a portion of the upper leg members 124A and 124B extend/rest along the base 104. In addition, the lower leg members 126A and 126B of the exoskeleton 101 may drape over the base 104 and extend towards the footrest assembly 110, such that the boot 128A rests along the footrest 110A, and the boot 128B rests along the footrest 110B. In this configuration, the exoskeleton 101 is ready for transportation and to receive an individual. Any number or form of straps or belts (not shown) may be implemented to maintain the exoskeleton 101 in place relative to the apparatus 100 when desired.

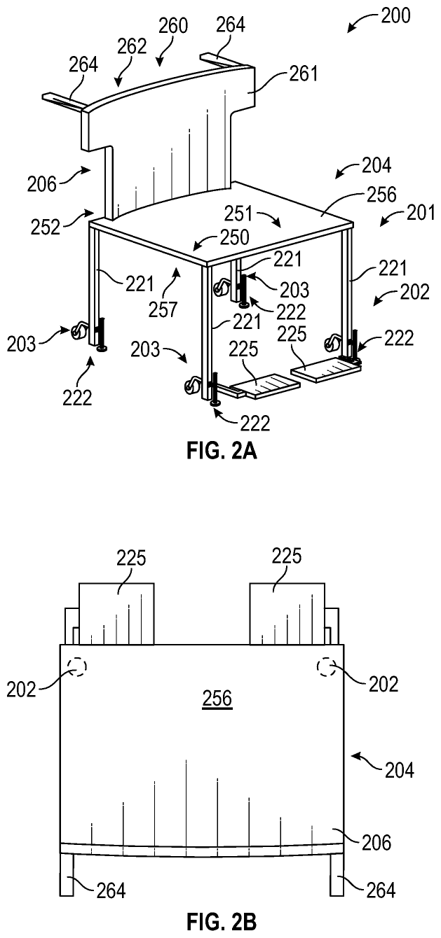

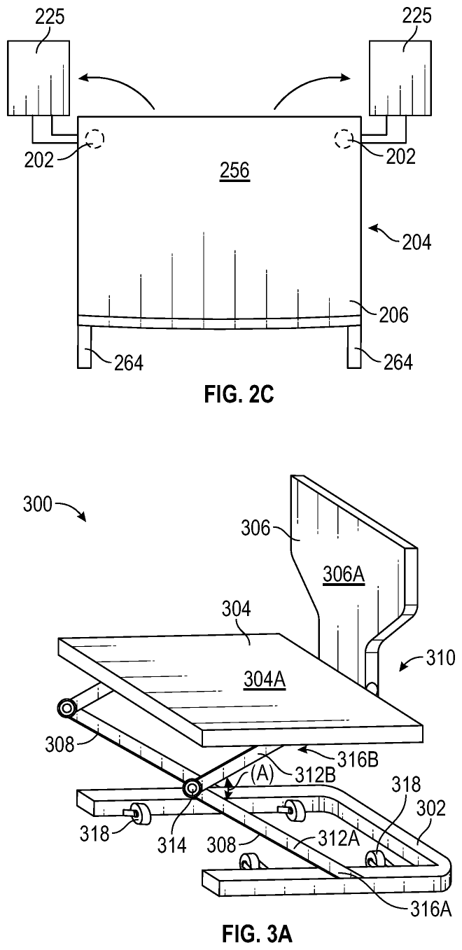

Referring to FIG. 2A, one embodiment of an apparatus 200 for storage and transportation includes a frame 201 comprising a plurality of legs 202, a seat 204, and a back 206 configured for transitioning between and locking in one or more configurations including a “transport” configuration optimized for the storage and transport of the exoskeleton, and a “sit-to-stand” configuration optimized for seating a patient within the exoskeleton during a physical therapy session. In some embodiments, a “display” configuration may be included in order to stand the exoskeleton up without the patient.

In some embodiments, each of the plurality of legs 202 comprises an elongated body 221 having an upper end 221A and a lower end 221B terminating in a wheel 203, and each of the plurality of legs 202 may further include a brake 222. In some embodiments, the plurality of legs 202 is divided into a pair of front legs 223 and a pair of rear legs 224, where each of the plurality of front legs 223 may include a footrest 225. The footrest 225 is operable for swinging laterally from a first position to a second position to aid with seating a patient within the exoskeleton. Alternatively, the footrest 225 may be retractable in any form to reduce the overall profile of the apparatus 200. The first position is shown in FIG. 2B, wherein the footrests 225 are individually oriented about a vertical axis defined by a direction of elongation of the legs 202 and are located directly in front of the seat 204. The second position is shown in FIG. 2C, wherein the footrests 225 are rotated about the vertical axis and are located lateral to the seat 204. The footrests 225 are operable for swiveling and locking into the first position for engagement with and storage of the exoskeleton in the “transport” configuration, and are also operable for swiveling and locking into the second position to create clearance for a patient's legs while the patient is putting on the exoskeleton or transitioning from a “sitting” position to a “standing” position in the “sit-and-stand” configuration.

Referring back to FIG. 2A, in some embodiments, the brakes 222 may be engaged in order to prevent the wheels 203 from rotating which temporarily maintains the apparatus 200 in a fixed position, thereby creating a stationary surface for the patient to sit on and transition into the exoskeleton using the apparatus 200. In some embodiments, engaging the brakes 222 may cause the wheels 203 to be lifted off the ground such that the brakes 222 contact the ground. In some embodiments, the brakes 222 may all be engaged or disengaged and the wheels 203 may all be disengaged or engaged simultaneously such that manual engagement or disengagement of each of the plurality of brakes 222 or the wheels 203 requires a single motion. The wheels 203 are suitable for transporting the exoskeleton using the apparatus 200 without the patient, but are also suitable for transporting the exoskeleton and patient together in case of an emergency. The brakes 222 may be engaged while the patient is sitting in the exoskeleton or while the patient is transitioning from a sitting position to a standing position using the exoskeleton. In some embodiments, each of the plurality of legs 202 comprises hardware enabling variable length to account for different sizes of exoskeletons which may be used with the apparatus 200.

In some embodiments, the seat 204 rests at the upper end 221A of each of the plurality of legs 202 and comprises a seat frame 250, wherein the seat frame 250 defines a front section 251, a rear section 252, a seat surface 256 and a seat underside 257, where the seat surface 256 is operable to receive an exoskeleton in the “sit-to-stand” or “display” configurations. In some embodiments, the seat 204 is 18 inches deep and 22 inches wide, and may be adjustable to accommodate different exoskeletons. In addition, the back 206 of the apparatus 200 is operably engaged with the rear section 252 of the seat frame 250. The back 206 comprises a back frame 260 defining a front surface 261 and a rear surface 262, wherein the front surface 261 engages with the exoskeleton in the “sit-to-stand” and “display” configurations and the rear surface 262 is operable to store extraneous exoskeleton hardware.

In some embodiments, the back 206 of the apparatus 200 further comprises a pair of handles 264 for pushing the apparatus 200 in the “sit-to-stand”configuration or in the “transport” configuration. In some embodiments, an additional handle (not shown) may be included on the apparatus 200 such that the apparatus 200 may be lifted and carried. The apparatus 200 may also include a locking mechanism (not shown) disposed along the back frame 260 or the seat frame 250 for locking the components of the apparatus 200 in the “transport” configuration, the “sit-to-stand” configuration, the “display” configuration, or any other configuration. In some embodiments, the back 206 of the apparatus 200 may further include a pouch or other form of storage compartment to store various tools or accessories for the apparatus 200.

In some embodiments, the back 206 and the seat 204 comprise a plurality of eccentric spaces 242 formed along the front surface 261 and the seat surface 256. The plurality of eccentric spaces 242 are configured to provide clearance for any protruding parts of the exoskeleton while the seat surface 256 and front surface 261 provide support for the exoskeleton. In some embodiments, one or more retention belts (not shown) may be included on the back 206 or the seat 204 of the apparatus 200 in order to strap the exoskeleton onto the apparatus 200. The seat surface 256 of the seat 204 or the front surface 261 of the back 206 may be comprised of a sturdy material much like carpet or vinyl to provide a non-slip surface that resists tearing and retains the shapes of the eccentric spaces 242. In other embodiments, the seat surface 256 of the seat 204 or the front surface 261 of the back 206 may be comprised of hard molded plastic. The seat surface 256 of the seat 204 or the front surface 261 of the back 206 may be easily removable from the seat frame 250 or the back frame 260 for replacement in case the seat surface 256 or the front surface 261 become damaged over time. In some embodiments, the frame 201 may be configured to support at least a 300-lb person plus the exoskeleton (usually about 50-100 pounds) in order to allow both the patient and the exoskeleton to safely be supported by the apparatus 200.

The frame 201 may further include a plurality of hinges (not shown) and the locking mechanism installed along the various components of the frame 201 in order to fold the back 206, the seat 204 and the legs 202 into the different configurations. In some embodiments, the frame 201 may be folded into the “transport” configuration when the additional handle is lifted. In other embodiments, the frame 201 may be wheeled around in the “sit-to-stand” configuration using the handles 264 and the wheels 203 with or without the exoskeleton. The “sit-to-stand” configuration also allows the brakes 222 to engage thereby allowing a patient to sit in the exoskeleton on the apparatus 200 while the brakes 222 are engaged. The “display” configuration may be any configuration of the components of the apparatus 200 which stands the exoskeleton up or seats the exoskeleton for display. Lastly, the “transport” configuration may also allow the components of the apparatus 200 to be folded into them such that a total volume of the apparatus 200 and exoskeleton are reduced or formed into a shape which is easy to store.

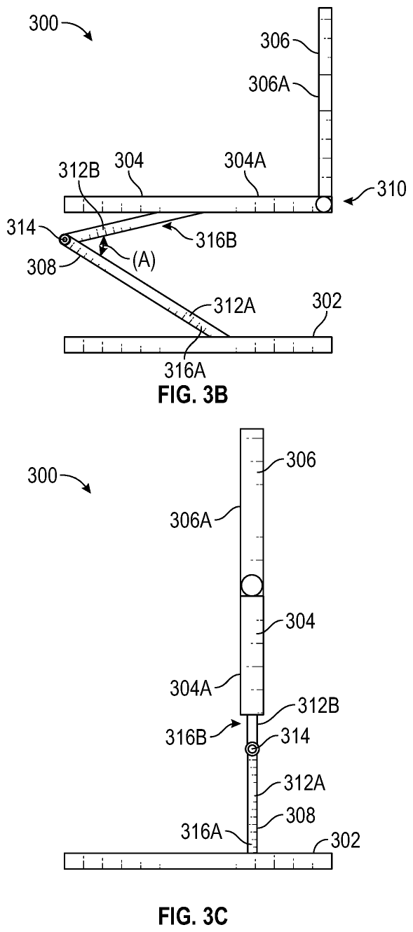

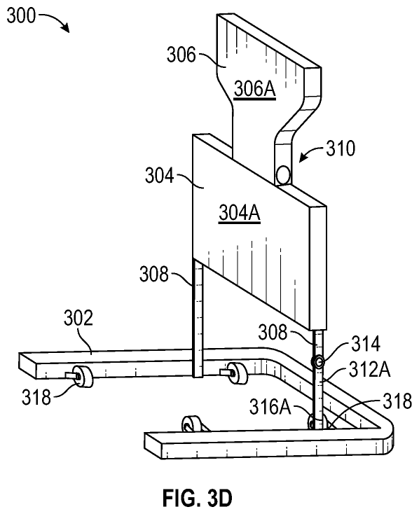

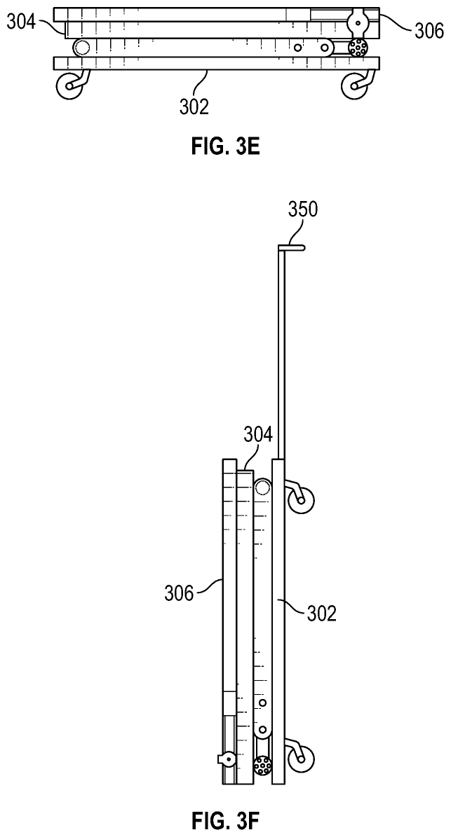

Referring to FIG. 3A, another embodiment of an apparatus, designated apparatus 300, for storage and transportation of an exoskeleton is shown. In general, the apparatus 300 includes a base 302, a seat 304, and a back 306 with the seat 304 generally positioned between the base 302 and the back 306. The seat 304 defines a seat surface 304A and the back 306 defines a back surface 306A configured for engagement with a portion of an exoskeleton (e.g., exoskeleton 101) and optionally an individual donning the exoskeleton. Further, the back 306 of the apparatus 300 is operably engaged with the seat 304 such that the back 306 may be folded over, into, or otherwise repositioned relative to the seat 304 and base 302 to reduce an overall profile size of the apparatus 300 in a collapsed or folded configuration, as further described herein. In some embodiments the apparatus 300 includes a locking mechanism that locks or transitions one or more of the aforementioned components to or from a storage/transport configuration or a deployed configuration.

As indicated, a plurality of legs 308 extends from the base 302 and generally interconnects the base 302 with the seat 304. In other words, the seat 304 is mounted to the plurality legs 308 opposite the base 302 such that the plurality of legs 308 suspends the seat 304 over the base 302 in a deployed or storage configuration. In some embodiments, a joint 310 such as a hinge or hinge portion of the seat 304 connects the back 306 with the seat 304 so that back 306 can rotate about the joint 310 relative to the seat 304 or vice versa (as indicated in FIGS. 3B-3D). In some embodiments, the base 302 is U-shaped as shown to reduce structure and provide mechanical support.

In some embodiments, each of the plurality of legs 308 defines an elongated member comprising at least two sections shown as 312A and 312B in communication with and bendable/foldable about a joint 314, such as a hinge. In particular, the section 312A extends from the base 302 and is interconnected, via the joint 314, with the section 312B that is coupled to the seat 304. In a deployed or seated configuration as is shown in FIG. 3A, the connection between the first section 312A and the second section 312B, defines an Angle (A) at the intersection, which may take the value of anywhere between 0 to 180 degrees (approximately 60 degrees in FIG. 3A).

As further shown, the first section 312A defines a first terminal end 316A of each of the plurality of legs 308 coupled to the base 302, and the second section 312B defines a second terminal end 316B of each of the plurality of legs 308 coupled to the seat 304. In some embodiments, the apparatus 300 is configured such that the first section 312A may be folded relative to the second section 312B along the joint 314, to bring the second terminal end 316B closer to the first terminal end 316A (to e.g., assume a folded or transport configuration). This action may be reversed in the event it is desired to move the seat 304 to a position farther way from the base 302, for whatever reason. Intuitively, the angle A changes as the position of the second terminal end 316B is changed relative to the position of the first terminal end 316A. The plurality of legs 308 may take the form of any variety of shapes suitable for positioning the seat 304 a predetermined height (which may be adjustable) above the ground/surface, and suitable for supporting a predetermined exoskeleton weight, and possibly a weight of a human in combination with the weight of an exoskeleton. The plurality of legs 308 may include any number or type of individual discrete sub-members foldable relative to one another along any number of hinges or other joints; however it is contemplated that a single contiguous member component may be employed for some applications.

As further shown in FIG. 3A, the apparatus 300 may include a plurality of wheels 318. The wheels 318 may include caster wheels, wheelchair wheels, or any standard wheel configuration. The wheels 318 may be retractable or adapted to swivel to an engaged position such that the wheels are oriented down towards the floor, or retracted or swiveled to the position shown in FIG. 3A during storage. In some embodiments, the plurality of wheels 318 may include retractable wheels including rubber casing and a retracting mechanism such that a caster of the wheel can be raised to engage a locking mechanism, and the caster may be lowered to deploy the wheel below the rubber casing to engage a surface/floor for rolling.

In a deployed or storage configuration, the back 306 extends perpendicularly from the seat 304 as shown, and the plurality of legs 308 are at least partially unfolded to suspend the seat 304 over the base 302 and so that the apparatus 300 may support a trunk or frame of an exoskeleton. In some embodiments, the base 302, seat 304, and the back 306 may define a general rectangular shape, and may be at least partially planar to accommodate engagement with various components of an exoskeleton. Other shapes are contemplated depending on the functionality of the apparatus 300 and how the components of the apparatus 300 engage or fold.

In a folded or transport/storage configuration (FIGS. 3C-3F), the components of the apparatus 300 may be re-positioned from the configurations of FIG. 3A and FIG. 3B to the configurations of FIGS. 3C-3D or FIGS. 3E-3F. In particular, the sections 312A and 312B defined by each of the plurality of legs 308 may be compressed, folded, re-positioned, or collapsed, so as to lower the seat 304 directly over the base 302 in the manner indicated in FIG. 3E. In general, as indicated in the transitions from FIGS. 3C-3D through FIGS. 3E and 3F, the apparatus 300 is configured to fold/collapse so that the seat 304 within a footprint of, over, or directly along the base 302. In one embodiment, the base 302, seat 304, and back 306 may be repositioned to form the configuration of FIG. 3E, such that the sea 304 is stacked over the base 302, and the back 306 is stacked over the seat 304. As shown in FIG. 3F, a handle 350 may be mounted to and retractable/foldable along the back 306 to navigate the apparatus 300 in the folded/transport configuration shown. In addition, a set of wheels may be engaged to the base 302. The wheels (not shown) may be retractable, removable, and/or otherwise configured to engage a stationary surface during transport and lock or retract when the apparatus is in a deployed configuration (e.g., FIG. 3A) when an exoskeleton is being applied to the apparatus 300.

While not shown, the apparatus 300 may include any other components that may facilitate transportation and storage of an exoskeleton, including, but not limited to a storage compartment for storing removable portions of an exoskeleton or tools for assembly thereof, and the like. A cushion material, which may be tear-resistant (not shown), may be mounted along the seat 304 and the back 306 to provide comfort to an individual utilizing the apparatus 300 to don an exoskeleton.

The apparatus 300 may include any number of joints or hinges to facilitate manipulation of the apparatus 300 to a folded or collapsed configuration shown in FIGS. 3A-3F. For example, the back 306 may be configured to fold flat along the base 302, and the legs 308 may be configured to fold along the bottom of the base 302 opposite the back 306. In addition, the apparatus may include a footrest assembly (not shown) that may be foldable relative to the plurality of legs 308. In other words, the footrests of FIG. 2 may be included with the apparatus 300.

In the configuration shown, the apparatus 300 may generally define a general chair/wheelchair shape configuration. However, the apparatus 300 may include any number or arrangement of configuration change components such as electric motors, pneumatics, levers, hydraulics, or actuators to facilitate movement or rotation of the components of the apparatus 300 relative to one another. For example, in some embodiments, the plurality of legs 308 may be rotatable, foldable, extendable and/or retractable using any number of actuators, mechanically, and/or electromechanically, to aid with seating of an individual along the apparatus 300. The apparatus 300 may include electrical components interconnected by any number of conductive members (not shown), or may be devoid of electrical components to reduce the weight and complexity of the apparatus 300 and/or facilitate folding.

Numerous related features are contemplated. For example, the apparatus 100 may comprise aluminum, titanium, composites, carbon fiber, or any number or combination of such materials to provide a lightweight yet durable structure. In some embodiments, aspects of the apparatus 300 may be formed using tubing (e.g., Comodita Brava rollator tubing) with an internal support, such as an I-beam. A seat cover may be formed along the apparatus 300 and may comprise Dyneema fabric or other similar material, and may be replaceable. In general, the apparatus 300 is configured to support at least a 220 pound person and an exoskeleton ranging from 10 to 100 pounds.

In some embodiments, the apparatus 300 is configured to receive an exoskeleton for enhanced storage and transportation thereof; and when engaged to the apparatus 300, the exoskeleton is more easily donned by an individual for use. An exoskeleton may take different forms and include different features or components as would be appreciated by those of ordinary skill the subject technical art.

It should be understood from the foregoing that, while particular embodiments have been illustrated and described, various modifications can be made thereto without departing from the spirit and scope of the invention as will be apparent to those skilled in the art. Such changes and modifications are within the scope and teachings of this invention as defined in the claims appended hereto.

What is claimed is:

1. An apparatus for storage and transport of an exoskeleton, comprising:
a base;
a seat positioned over the base;
a back mechanically coupled to the seat; and
a plurality of legs interconnecting the seat with the base,
wherein the seat and the back are configured for engagement with an exoskeleton, and
wherein the seat, the back, and the plurality of legs are adapted to transition between and temporarily maintain one of a plurality of configurations.

2. The apparatus of claim 1, wherein the apparatus further comprises:
a locking mechanism, wherein the locking mechanism is operable for locking the seat, the back, and the plurality of legs into one of the plurality of configurations.

3. The apparatus of claim 1, further comprising:
a plurality of wheels coupled to the base; and
a plurality of brakes in operative engagement with the plurality of wheels, with each of the plurality of brakes being operable to be engaged or disengaged simultaneously.

4. The apparatus of claim 1, further comprising:
a plurality of wheels coupled to the base, each of the plurality of wheels being retractable towards in a vertical direction towards the back when the apparatus is in a storage or deployed configuration.

5. The apparatus of claim 1, further comprising:
one or more handles defined along a rear surface of the back for transporting the apparatus.

6. The apparatus of claim 1, further comprising:
one or more retention belts positioned along the seat and the back for engagement with the exoskeleton.

7. The apparatus of claim 1, further comprising:
a plurality of footrests positioned along the base, each of the plurality of footrests being adapted to transition between a first position and a second position.

8. The apparatus of claim 7, wherein the first position is defined directly in front of the seat and wherein the second position is defined lateral to the seat, each of the plurality of footrests being adapted to swivel between the first position and the second position.

9. The apparatus of claim 1, wherein each of the plurality of legs define an elongated member comprising at least two sections in communication via a joint, the at least two sections adapted to fold about one another by way of the joint along a horizontal axis extending along a lateral side of the seat.

10. The apparatus of claim 9, further comprising one or more pneumatic shocks for engaging the plurality of legs, wherein the at least two sections of the elongated member are adapted to transition from a first configuration to a second configuration by assistance from the one or more pneumatic shocks.
